(19) United States
(12) Patent Application Publication
(10) Pub. No.: US 2004/0142946 A1
(43) Pub. Date: Jul. 22, 2004

(54) Modified nucleosides and nucleotides and use thereof

(76) Inventor: Jyoti Chattopadhyaya, Uppsala (SE)

Correspondence Address: YOUNG & THOMPSON, 745 SOUTH 23RD STREET 2ND FLOOR, ARLINGTON, VA, 22202

(21) Appl. No.: 10/399,951

(22) PCT Filed: Nov. 9, 2001
(86) PCT No.: PCT/SE01/02484

(51) Int. Cl. A61K 31/52; A61K 31/513; C07D 473/14

(52) U.S. Cl. 514/263.2; 544/276; 544/277; 544/317; 514/269

(57) ABSTRACT

The present invention relates to modified nucleotides and nucleosides and reagents to produce these. The modified nucleotides and nucleotides are assembled to larger oligonucleotides and oligonucleosides, which, for example, may be used for diagnostics of polymorphisms and for antisense therapy of various conditions. The oligonucleotides and oligonucleosides described in the invention have very good endonuclease resistance without compromising the RNA cleavage properties of RNase H wherein combinations of modifications with Y, Z, R or B are claimed: X=O or S, NH or NCH₃, CH₂ Or CH(CH₃), Y=O, S, or NH or NCH₃, CH₂or CH(CH₃); Z=O, S, or NH or NCH₃, CH₂ or CH(CH₃); R=O or S, or NH or NCH₃, CH₂ or CH(CH₃); B=A, C, G, T; 5-F/cl/BrU or —C, 6-thioguanine, 7-deazaguanine; α- or β-D- (or L)ribo, xylo, arabino or lyxo configuration.

### FIELD OF THE INVENTION

The present invention is within the field of molecular biology. More closely, it relates to modified nucleotides and nucleosides and the use thereof as building blocks for incorporation into oligonucleotides and oligonucleosides. These may, for example, be used for antisense therapy.

### BACKGROUND

The recruitment by RNase H, an endogenous enzyme that specifically degrades target RNA in the antisense oligonucleotide (AON)/RNA hybrid duplex is an important pathway for the antisense action beside the translational arrest. RNase H hydrolyses the RNA strand in an RNA/DNA hybrid in a catalytic manner. It produces short oligonucleotides with 5′-phosphate and 3′-hydroxy groups as final products. Bivalent cations as Mg²⁺ and Mn²⁺ are found to be necessary cofactors for enzymatic activity. The enzyme is widely present in various organisms, including retroviruses, as a domain of the reverse transcriptase. The RNase H1 from Escherichia coli is the most characterized enzyme in this family.

RNase H promoted cleavage of the viral mRNA via formation of the duplexes with complementary oligo-DNAs (antisense strand) is one of the strategies to treat pathogen infections and other genetic disorders. Recent isolation of the human RNase H1 and RNase H2 highlights the importance of the development of the antisense drugs utilizing this mechanism of action.

It has been suggested that for eliciting the RNase H in AON/RNA hybrid, the AON part should retain the B-type DNA conformation with 2′-endo sugar (South-type, S), while the RNA moiety should retain its A-type helix character with 3′-endo sugar (North-type, N). To fulfill these requirements various modifications of sugar, base as well as of the phosphate backbone have been attempted and numerous reports are available about these modified AONs and their antisense action. Among these, AONs having one or more conformationally fixed (either in N- or S-form of the sugar pucker) nucleoside residues have been found to be promising candidates because when they are locked in the N-form, they exhibit high affinity to the target RNA. Recently, the locked nucleic acid (LNA), in which the sugar moiety is fixed in the North conformation, has shown unprecedented affinity towards RNA. LNA and other modifications which have the fixed N-sugar moiety drive the AON helix to the A-type resulting in RNA/RNA type duplex which accounts for their higher binding affinity, but this leads to the loss of RNase H action. The introduction of conformationally constrained N-methanocarba-thymidine residue in the N-form increased the thermodynamic stability of AON/RNA duplex, whereas in the S-form, a destabilizing effect was observed. It was later found that multiple introduction of (N)-methanocarba-thymidines, although increased the thermodynamic stability of the AON/RNA duplex, but failed to recruit any RNase H activity. It is now quite clear that all modifications that lead to preferential North-type sugar, including its constrained form, in an RNA-type AON result in the loss of RNase H activity, because they resemble RNA/RNA duplex, except when they appear at the termini or in the middle in the gapmer-AON. It has been so far assumed that probably three or four N-type conformational repeats are necessary to enhance the thermal stability of RNA-type AON/RNA duplex. Nobody however specifically knows how many North-constrained nucleosides are required to alter the conformational tolerance of the RNase H recognition, thereby its substrate specificity, owing to the local structural perturbations in an RNA-type AON/RNA hybrid. On the other hand, 2′-methoxy, 2′-F or 2′-O—CH₂—CH₂—OCH₃ based (and other analogous) antisense chemistry, used as a gapmer, promote RNA cleavage by RNase H at least three-fold less satisfactorily than the native. These 2′-O-alkoxy substituted nucleotides are incorporated in the antisense strand as a gapmer to promote complementary RNA cleavage by RNase H. These work better than many other compounds that are available in the literature, but they work less satisfactorily than the native in terms of RNA cleavage efficiency. The efficiency of these 2′-O-methoxy, 2′-F or 2′-O—CH₂—CH₂—OCH₃ based gapmers, “without exception cleaved at slower rate than the wild type substrate” (Crooke et al, Biochemistry, 36, p390-398 (1997)); they work (catalytically) at about 3-fold less efficiency as that of the native counterpart.

Arabino nucleic acids (ANA) have been recently tested for their ability to activate RNase H. Both the sequences tested had lower thermodynamic stability in comparison with the natural DNA/RNA hybrid duplex. CD spectra of these duplexes showed close resemblance to the native DNA/RNA duplexes. Although no quantitative data available, the duplexes formed by ANA and complementary RNA were found to be poorer substrates for RNase H assisted cleavage compared to the native counterpart. However when Mn²⁺ was used instead of Mg²⁺ in the reaction medium, nearly complete degradation of the target RNA was observed. The 2′F-ANA has also been explored for RNase H potency. Their hybrids with RNA showed higher Tₘ than the native DNA/RNA hybrid duplex (ΔTₘ=+5° C.) and also exhibited global helical conformation similar to native DNA/RNA hybrids as revealed by CD spectroscopy. RNase H promoted cleavage of these 2′F-ANA/RNA hybrids were found to be similar to that observed for native DNA/RNA and DNA-thioate/RNA hybrids. No endonuclease resistance properties of these 2′F-ANA are however known.

Recently, cyclohexenyl nucleosides have been incorporated to AONs (CeNA), and found to have stabilizing effect with the target RNA. The CD spectra of CeNA/RNA hybrid showed close resemblance to the native counterpart. Incorporation of one, two, or three cyclohexenyl-A nucleosides in the DNA strand increases duplex stability with +1.1, +1.6, and 5.2° C. The stabilization effect as expected also depends on the site of introduction. But when tested for RNase H activity they were found to be a relatively poorer substrate for the enzyme in comparison with the native.

Boranophosphate oligothymidines (11mer borano-AON where one of the nonbridging oxygens is replaced with borane) were reported to support RNase H hydrolysis of poly(rA) with efficiency higher than non-modified thymidine oligos regardless of their poor affinity towards the target RNA. The borano modification produces minimal changes in the CD spectrum of the thymidine dimer compared to the native counterpart and both diastereomers adopt B-type conformation (the same as unmodified d(TpT) dimer). Unfortunately, there is no CD or any other structural data available on the hybrid duplexes of such borano-AONs with RNA, which makes it impossible to assess the structural background for the recognition of these duplexes as the substrates by the RNase H vis-à-vis natural counterpart.

Chimeric methylphosphonate based antisense oligos with 5-4-5 methylphosphonates-phosphate-methylphosphonates construct, in particular, having a Tₘ of about 37° C., was at this temperature more than 4-fold effective at eliciting RNase H hydrolysis of mRNA than the natural congener of Tₘ 51° C.

### SUMMARY OF THE INVENTION

The substituted antisense oligonucleotides according to the invention, although show a drop of Tₘ compared to the native counterpart, can recruit RNase H to cleave the complementary RNA at least as efficiently as the native. The engineering of 3′-exonuclease resistance is rather easily achieved by several means but it is rather difficult to engineer endonuclease resistance without sacrificing on the binding properties to the complementary RNA, or the RNA cleavage by RNase H. The present invention, on the other hand, can combine both of these properties (i.e. RNase H mediated cleavage of the complementary RNA strand, as well as the endonuclease resistance of the antisense strand). For example triple oxetane modified oligos show at least four times better endonuclease resistance to the antisense oligos without compromising any RNA cleavage property by RNase H, compared to the native counterpart.

The present inventors have found that the minor groove in AON/RNA duplexes should fulfill following requirements: (1) 1,2-constrained nucleoside derivatives when incorporated in to the AON give the corresponding AON/RNA duplex preferred helical structure such that the minor groove can accommodate the chemistry of the RNase H cleavage (cleavage site should at least have one B-type DNA conformation in the AON strand with the A-type conformation in the complementary RNA, as suggested by our engineering of the single-point RNA cleavage reaction by RNase H). (2) Such AON/RNA heteroduplexes should be also adequately flexible (as seen by the characteristic lower Tm values, compared to the native counterpart) to accommodate the conformational change required upon complexation with RNAse H—Mg²⁺ in the minor groove for the RNA cleavage by RNase H. (3) The modifications in the minor groove or in its proximity, brought about by a specific 1,2-fused systems in to AON/RNA hybrids do not significantly alter the hydration pattern and secures the availability of the 2′-OH of the RNA for interaction with the active site of RNAse H and Mg²⁺.

In a first aspect, the present invention relates to modified nucleosides and nucleotides, enabling five-membered sugars or their derivatives to be conformationally constrained in the North/East region of the pseudorotational cycle, represented by the following formula:

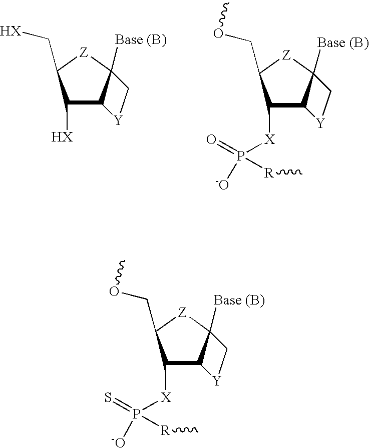

wherein combinations of modifications with X, Y, Z, R or B are claimed:

X=O or S, or NH or NCH₃, CH₂ or CH(CH₃),

Y=O, S, or NH or NCH₃, CH₂ or CH(CH₃);

Z=O, S, or NH or NCH₃, CH₂ or CH(CH₃)

R=O or S, or NH or NCH₃, CH₂ or CH(CH₃)

B=A, C, G, T, U, 5-F/Cl/BrU or —C, 6-thioguanine, 7deazaguanine;

α- or β-D- (or L) ribo, xylo, arabino or lyxo configuration

In a second aspect the invention relates to reagents for the preparation of modified oligonucleotides and oligonucleosides by solid or solution phase synthesis:

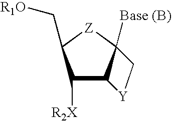

wherein combinations of modifications with Y, Z, R or B are claimed:

X=O or S, or NH or NCH₃, CH₂ or CH(CH₃),

Y=O, S, or NH or NCH₃, CH₂ or CH(CH₃);

Z=O, S, or NH or NCH₃, CH₂ or CH(CH₃)

R=O or S, or NH or NCH₃, CH₂ or CH(CH₃)

B=A, C, G, T, U, 5-F/Cl/BrU or —C, 6-thioguanine, 7-deazaguanine;

α- or β-D- (or L) ribo, xylo , arabino or lyxo configuration

R₁=5′-protecting group according to claim 2.

R₂=3′-phosphate, 3′-(H-phosphonate), 3′-phosphoramidate, 3′-phosphoramidite, 3′-(alkanephosphonate) according to claim 2.

The different bases, B, may be varied as in claim 2.

In a third aspect, the invention relates to oligonucleotides and oligonucleosides comprising the above modified compounds. These modified monomer blocks according to the invention are introduced (1-9 units) in, for example, antisense oligonucleotides for site-specific modifications, depending upon the length Thus, the invention provides novels antisense oligos, AON's. The native nucleotides are fully or partly substituted in the antisense strand by the modified analogs according to the invention.

The oligoribonucleotides and oligoribonucleosides can include substituent groups (both in the tethered and non-tethered form) for modulating binding affinity or artificial nuclease activity to the complementary nucleic acid strand as well as substituent groups for increasing nuclease resistance and for RNase H promoted cleavage of the complementary RNA strand in a site-specific fashion. The oligomeric compounds are useful for assaying for RNA and for RNA products through the employment of antisense interactions, and for the diagnostics, for modulating the expression of a protein in organisms, detection and treatment of other conditions and other research purposes, susceptible to oligonucleotide therapeutics. Synthetic nucleosides and nucleoside fragments are also provided useful for elaboration of oligonucleotides and oligonucleotide analogs for such purposes.

This invention relates for example to compounds based on the oligomeric compounds containing one or more units of 1′,2′-fused oxetane, 1′,2′-fused azatidine, 1′,2′-fused thiatane or 1′,2′-fused cyclobutane systems with pentofuranose or the cyclopentane moieties or with any other endocyclic sugar modified (at C4′) derivatives (thereby producing North-East) (N/E) conformationally constrained nucleosides), in either oligonucleotide or oligonucleoside form. These conformationally-constrained nucleosides and nucleotide derivatives (in the N/E constrained structures) in the oligomeric form, when form basepaired hybrid duplexes with the complementary RNA strand, can be useful for modulating the activity of RNA in the antisense therapy or DNA sequencing, in the diagnosis of the postgenomic function or in the design of RNA directed drug development.

In a fourth aspect, the invention relates to therapeutic composition comprising the modified oligonucleotides and oligonucleosides above together with physiologically acceptable carriers.

The main therapeutic use of the composition is antisense therapy of, for example, oncogenic and pathogenic sequences and genetic disorders. Another therapeutic use is to incorporate these blocks into Ribozyme (Catalytic RNA) in order to cleave the target RNA. These blocks can be transformed by nucleoside kinases to the triphosphate form by serving as acceptors from the phosphate donors such as ATP or UTP (J. Wang, D. Choudhury, J. Chattopadhyaya and S. Eriksson, Biochemistry, 38, 16993-16999 (1999). Because of their broader substrate specificities, these triphosphates can interfere with the DNA synthesis of various pathogen and oncogen (antivirals and antitumors).

In a fifth aspect, the invention relates to a diagnostic kit comprising the modified oligonucleotides and oligonucleosides as defined above.

The diagnostic kit is mainly intended for detection of single nucleotide polymorphism SNP and multiple nucleotide polymorphisms MNP. The diagnostic kit is for in vitro use on a human body sample, such as a blood sample. See the following website: http://www.genetrove.com/ of antisense technology for gene functionalization and target validation using 2′-O-alkyl based antisense technology, which is applicable (albeit more efficiently) with the present invention: 1,2-fused sugar technology.

Regulation of how and when genes are turned into proteins can occur at several levels, but RNA is by far the most important generator of complexity and has an enormous potential for creating variation because this go-between molecule stands at the crossroad between genes and proteins. The 1,2-fused system when incorporated in the antisense strand (the antisense technology with the help of RNase H) can be used for systematic studies of how an organism regulates this flexibility through the RNA synthesis and processing (splicing). Thus the antisense technology, using the 1,2-sugar fused nucleoside based chemistries (see the above Figure), is highly relevant to functional genomics—specifically, gene functionalization and target validation, which, in turn to facilitate the discovery and development of new drugs.

In a sixth aspect, the invention relates to a DNA sequencing kit comprising the modified oligonucleotides and oligonucleosides as defined above.

The standard Sanger's dideoxynucleotide sequencing strategy using DNA polymerase and the 2′,3′-dideoxynucleotide triphosphates is used (see: http://www.accessexcellence.org/AE/newatg/Contolini/). See also the following website for details of the dideoxynucleotide sequencing strategy: http://www.ultranet.com/˜jkimball/BiologyPages/D/DNAsequencing.html

Under the procedure in the website, the 5′-triphosphate building blocks of 1,2′-fused-3′-deoxy-nucleoside (shown below)

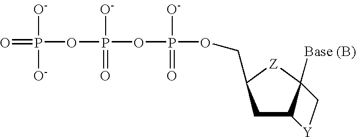

wherein combinations of modifications with Y, Z, or B are claimed:

Y=O, S, or NH or NCH₃, CH₂ or CH(CH₃);

Z=O, S, or NH or NCH₃, CH₂ or CH(CH₃)

B=A, C, G, T, U, 5-F/Cl/Br-U; 7-deaza-G or hypoxanthine

α- or β-D-(or L) ribo, xylo , arabino or lyxo configuration

are used instead of the standard 2′,3′-dideoxynucleotide 5′-triphosphates. The use of 7-deza-guanine or hypoxanthine analog considerably. reduce the aggregation owing to the weaker basepairing with dCTP, which, in turn, helps to reduce “compression artifacts” in sequencing gels: http://www.usbweb.com/products/reference/index.asp?Toc_ID=8

In a seventh aspect, the invention relates to use of the modified nucleotides and nucleosides of the invention to produce aptamers (using SELEX procedures, see for example the following website: http://www.somalogic.com/) comprising the modified oligonucleotides and oligonucleosides as defined above. The aptamers may consist of one or several 1,2-modified nucleosides, as defined above, which bind directly to the target proteins or any other ligand, inhibiting their activity.

In an eighth aspect, the invention relates to use of the modified nucleosides, nucleotides and their oligomeric forms of the invention for drug development or in any form of polymerase chain reaction (PCR) or in any molecular biology kit for diagnosis, detection or as reagent.

The present invention was based on the following observations:

1. The introduction of one to five units (North-East) (N/E) conformationally constrained nucleoside(s), such as [1-(1,3′-O-anhydro-β-D-psicofuranosyl)thymine] (T), see claim 1 for a full list, in to an antisense (AON) strand does not alter the global helical structure of the corresponding AON/RNA hybrid as compared to the native counterpart.

2. Despite the fact that a series of one to five units of N/E-constrained modified AON/RNA hybrid duplexes showed a drop of 2-6° C./modification in Tₘ (depending upon the number of 1,2-constrained A, C, G or T moieties in the antisense oligo and the composition of sequence), they were cleaved by RNase H with comparable efficiency (or better) as compared to the native counterpart.

3. It was also found that the target RNA strand in the hybrid AON/RNA duplex was resistant up to 5 nucleotides towards 3′-end from the site opposite to the introduction of the N/E-constrained unit in the AON strand, thereby showing the unique transmission of the N/E-constrained geometry of the N/E-constrained residue through the hybrid duplex (i.e. the 5-basepaired region has a putative RNA/RNA type duplex structure). An appropriate placement of two such N/E-constrained residues in the AON strand can thus produce a single cleavage site in the complementary RNA strand by RNase H.

4. Despite the fact that some of these sugar-modified AON/RNA duplexes (with three modifications, for example) were destabilized by up to 20° C. compared to the native counterpart, they were found to be as good substrate for RNase H as the native hybrid duplex. The RNase H recruiting power of the oxetane-locked or similarly fused thiatane, azatidine or AONs/RNA hybrids suggests the importance of kinetics as well as relationship between the thermodyanamics of stability/flexibility of hybrid duplexes and the structure/dynamic vis-à-vis recognition, structural tolerance of the hybrid duplex-RNase H complex. Clearly, AON/RNA hybrids should possess certain degree of structural flexibility to undergo certain conformational readjustments upon complexation with RNase H and Mg²⁺ in the minor groove, which is necessary for the cleavage reaction. Those hybrid duplexes which are highly stable have poor conformational flexibility, and are not capable of structurally adjusting themselves upon complexation to the RNase H and Mg²⁺ to form an activated complex to give the cleavage reaction. This is why RNase H do not hydrolyse (or very poorly hydrolyze) those AON/RNA hybrid duplexes which are very stable. Since the RNase H cleavage of the complementary RNA is a slower process than the self-assembly of the AON/RNA hybrid, a smaller population of the hybrid duplex might be actually adequate to bind to RNase H and drive the complementary RNA cleavage to completion, thereby showing the importance of competing kinetics in the overall cleavage reaction This is expected to be the case under a non-saturation condition for hybrid duplexes with relatively low Tₘ as in our oxetane- (or other similarly) modified fused systems.

5. The thermodynamic instabilities of 1,2-fused sugar-modified (i.e. N/E-constrained) AONs/RNA hybrids were partially restored by the introduction of dipyridophenazine (DPPZ) moiety at the 3′-end (or at the 5′-end) of these AONs, which also gave enhanced protection towards 3′-exonucleases, and showed equally good RNase H cleavage property as the native counterpart. This was also applied to other 3′-substituents such as cholic acid, folic acid and cholesterol derivatives. AR of these tethered substituents were found to be non-toxic in various cellular assays.

6. The loss in the thermodynamic stabilities of 1,2-fused sugar-modified (i.e. N/E-constrained) AONs/RNA hybrids with the corresponding oxetane-modified C and G derivatives is ca 2-2.5° C./modification. The actual thermodynamic stability of a given antisense oligo thus depend on the number and type of 1,2-fused sugar-modified A, C, G or T or any other nucleotide blocks

7. The sugar-modified AONs were found to have 3-9 fold more endonuclease resistance compared to those for the native counterparts.

### DETAILED DESCRIPTION OF THE INVENTION

### BRIEF DESCRIPTION OF THE DRAWINGS

The numerous objects and advantages of the present invention may be better understood by those skilled in the art by reference to the accompanying figures, in which:

FIG. 1 shows the chemical structure of modified T thymine ([1-(1′,3′-O-anhydro-β-D-psico-furanosyl)thymine).

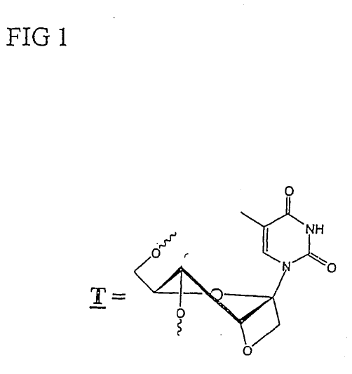

FIG. 2 shows a typical synthetic scheme for the preparation of oxetane-fused nucleosides according to the invention. The following reagents were used: (i) 4-toluoyl chloride, pyridine, r.t., overnight; (ii) silylated base, TMSOTf, acetonitrile, 4° C., 1 h, r.t., 18 h; (iii) Ms—Cl, pyridine, 4° C., overnight; (iv) 90% aqueous CF₃COOH, r.t., 20 min.; (v) NaH, DMF, 4° C., 9 h; (vi) methanolic NH₃, r.t., 2 days; (vii) DMTr-Cl, pyridine, r.t., overnight;(viii) 2-cyanoethyl-N,N-diisopropyl-phosphoramidochloridite, N,N-diisopropylethyl-amine, acetonitrile, r.t., 2 h.

The following observations give an insight in to the behavior of various T modified AON/RNA hybrids towards RNase H cleavage as well as their stability toward endo and exonucleases:

(1) The extent of RNA cleavage in hybrid duplexes by E. coli RNase H1 in the native hybrid [DNA/RNA] was found to be 68±3%. The target RNA with all single T, double T and triple T modified AONs, were hydrolyzed under the same conditions with extend of 51-68±3%.

(2) In the AON/RNA hybrid duplexes with a single mismatch, the RNA was cleaved at a comparable rate as the native counterpart although the hybrid shows a loss of 10-11° C. in Tₘ. owing to the mismatch. They also showed additional cleavage sites. These two observations therefore show that the recognition of the oxetane-based T vis-a-vis a mismatch in the AON strand by the target RNA is indeed different, most probably owing to the fact that T was perhaps partially hydrogen bonded

(3) The five nucleotide resistance rule to the RNase H cleavage of the RNA in the AON/RNA hybrids in all single T, double T and triple T modified AONs allowed us to engineer a single cleavage site in the target RNA by RNase H. The single RNA cleavage site has been earlier shown to occur in case of 2′-O-methyl modified chimeric AON/RNA duplex in which all the central 2′-deoxynucleotides except the middle nucleotide have been shown to adopt an RNA-type conformation by NMR spectroscopy. Since the CD spectra showed that all our T modified AON/RNA hybrid duplexes have global structure that corresponds to DNA/RNA type duplex (indicating that our AONs retain the B-DNA type helical conformation in the hybrid), we conclude that the 5-nucleotides resistance rule observed with our T modified AONs is owing to more subtle local microscopic conformational (and/or hydration) change, which is only detectable by the enzyme, not by the CD.

(4) The three T modified AONs gave the endonuclease stability (with DNase 1) almost 4 fold better (87% of AON remained after 1 h of incubation) compared to the natural counterpart (19% left), but their 3′-exonuclease stability was identical to that of the native AON. The 3′-exonuclease stability was however improved by using three T modifications along with the 3′-tethering of dipyridophenazine (DPPZ) moiety, in that 85% of AON was intact while the native AON was completely hydrolyzed after 2 h of incubation with SVPDE (note that the endonuclease resistance remained however unchanged). The RNase H promoted cleavage of this AON/RNA duplex (59±4%) remained very comparable to that of the counterpart with the native AON (68±3%) and with three T modified AON (61±6%), although a gain of 7° C. of Tₘ was achieved by this additional 3′-DPPZ modification. This again shows that the rise of Tₘ do not necessarily dictate the RNase H cleavage as was earlier found for some methylphosphonate chimeras and boranophosphates. It should be however noted that the presence of the 3′-DPPZ moiety produces an additional cleavage site. This is most probably owing to the stabilization of the terminal G-C hydrogen bonding by the 3′-DPPZ group (observed by NMR) as well as the recognition of the DPPZ by the enzyme both of which appears to be important for RNase H recognition, binding and cleavage. Interestingly, amongst all the T modified AONs studied so far, this is the only example where the 5-nucleotide resistance rule in the RNA strand is not obeyed.

Experimental Part

General Procedure for Preparation of Oxetane-Modified Antisense Oligonucleotides (AONs).

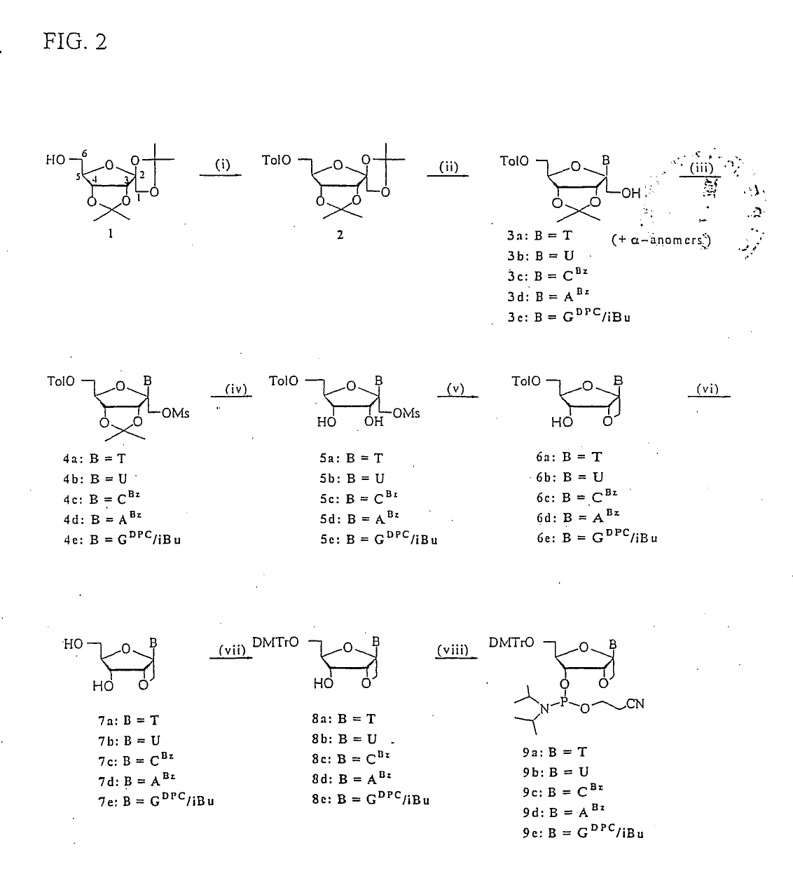

The title compound (7a) was prepared from 1,2:3,4-bis-isopropylidene-β-D psicofuranose (1) (FIG. 2) which was synthesized from D-fructose. Protection of 1 with 4-toluoyl group to give 2, which was coupled with O,O-bis(trimethylsilyl)thymine in the presence of TMSOTf as Lewis acid and acetonitrile as solvent to furnish (1:1) anomeric mixture of the protected psiconucleosides 3a (β-isomer) and the corresponding α-isomer in 67% yield. They were separated by careful column chromatography and the stereochemistry of C2′ in 3a was confirmed by means of NOE measurements. Methanesulfonylation of β-anomer 3a afforded 1′-mesylate 4a (98%) from which the isopropylidine was deprotected using 90% aqueous CF₃COOH to yield 5a (92%). The oxetane ring formation was achieved by treatment of 5a with NaH in DMF at 0° C. for 9 h to give 6a (60%). Removal of the 4-toluoyl group from 6a furnished the desired 1-(1′,3′-O-anhydroβ-D-psicofuranosyl)thymine (7a), which was converted to phosphoramidite building block 9a (90%) through 6′-O-4,4′-dimethoxytrityl derivative 8a. The phosphoramidite 9a was then used for incorporation of T residue into AONs (3)-(6). Similarly, phosphoramidates 9b-9e were purified and incorporated into various AONs.

Typical Experiments

6′-O-4-Toluoyl-1,2:3,4-bis-O-isopropyliene-D-psicofuranose (2).

The psicofuranose (1) (5.9 g, 22.5 mmol) was coevaporated with pyridine 3 times and dissolved in 100 ml of the same solvent. The solution was cooled in an ice bath and 4-toluoyl chloride (3.3 ml, 1.1 mmol) was added dropwise under nitrogen atmosphere. The mixture was stirred at the same temperature for 2 h. Saturated sodium bicarbonate solution was added and stirring was continued for further 2 h, and then extracted by DCM. The organic phase was washed with brine and dried over MgSO₄, evaporated and coevaporated with toluene. Recrystallisation from methanol furnished 2 (7.7 g, 20.2 mmol, 90%). R_{f}: 0.75 (System A). ¹H-NMR (CDCl₃): 7.9 (d, J=8 Hz, 2H), 4-toluoyl; 7.3 (d, J=7.9 Hz, 2H), 4-toluoyl; 4.8 (d, J_{3,4}=5.7 Hz, 1H), H-4; 4.7 (d, 1H), H-3; 4.48-4.35 (m, 3H), H-5, H-6, H-6′; 4.33 (d, J_{1,1′}=9.7 Hz, 1H), H-1; 4.1(d, 1H), H-1′, 2.41 (s, 3H), CH₃,4-toluoyl; 1.46 (s, 3H), 1.44 (s, 3H), 1.35, 1.33 (s, 2×3H) CH₃, isopropyl. ¹³C-NMR (CDCl₃): 166.3 (C═O, 4-toluoyl); 143.7, 129.8, 128.9, 126.8 (4-toluoyl); 133.6, 112.7, 111.6; 85.2 (C-3); 82.9 (C-5); 82.3 (C-4), 69.7 (C-1), 64.5 (C-6); 26.4, 26.2, 24.8 (CH₃, isopropyl); 21.2 (CH₃,4-toluoyl).

1-(3′,4′-O-Isopropyliene-6′-O-[4-toluoyl]-α-D-psicofuranosyl)thymine and 1-(3′,4′-O-isopropyliene-6′-O-[4-toluoyl]-β-D-psicofuranosyl)thymine (3a).

Thymine (3.7 g, 29.6 mmol) was suspended in hexamethyldisilazane (35 ml) and trimethylchlorosilane (5.6 ml) was added. The reaction mixture was stirred at 120° C. in nitrogen atmosphere for 16 h. The volatile material was evaporated and the residue was kept on an oil pump for 20 min. Sugar 2 (7.0 g, 18.5 mmol) was dissolved in dry acetonitrile and added to the persilylated nucleobase. The mixture was cooled to 4° C. and trimethylsilyl trifuromethanesulfonate (4.3 ml, 24 mmol) was added dropwise under nitrogen atmosphere. After being stirred at 4° C. for 1 h, the mixture was stirred at room temperature for 18 h. Saturated NH₄Cl was added to the reaction mixture and stirred for 30 min. The organic layer was decanted and the aqueous layer was extracted 3 times with ether. The combined organic phase was washed first with saturated sodium bicarbonate solution and then with brine. It was then dried over MgSO₄, filtered and evaporated. The resultant oil was carefully chromatographed using 0-3% MeOH-DCM yielding 3a and the corresponding α-anomer. 3a: (5.5 g, 12.3 mmol, 67%) R_{f}: 0.5 (System B). (α-anomer of 3a): ¹HNMR (CDCl₃): 8.8 (s, 1H), NH; 7.95 (d, J=8.2 Hz, 2H), 4-toluoyl; 7.5 (s, 1H), H-6; 7.28 (d, J=8.4 Hz, 2H), 4-toluoyl; 5.22 (d, J_{3′,4′}=5.9 Hz, 1H), H-3′; 4.83 (t, J_{4′, 5′}=4.7 Hz, 1H), H-4′; 4.71 (dd, J_{gem}=13.1 Hz, J_{5′,6′}=7 Hz, 1H), H-6′; 4.55-4.38 (m, 2H), H-5′, H-6″; 4.29 (dd, J_{gem=}11.8 Hz, J_{1′,1′OH}=7.9 Hz, 1H), H-1′; 3.79 (dd, J_{1″,1′OH}=6.7 Hz, 1H) H-1″; 3.34(t, 1H), 1′-OH; 2.43 (s, 3H) 4-toluoyl; 1.92 (s, 3H), CH₃; 1.39, 1.34 (s, 2×3H), CH₃. ¹³C-NMR (CDCl₃): 166.6 (C═O, 4-toluoyl); 164.1 (C-4); 150 (C-2); 144.3 (4-toluoyl); 135.1 (C-6); 129.6, 129.2, 126.2 (4-toluoyl); 113.8 (C-5); 108.9 (C—Me₂); 99.7 (C-2′); 83.7 (C-5′); 82.5 (C-3′); 80.7 (C-4′); 65.1 (C-1′); 63.7 (C-6′); 27, 25.3 (CH₃, isopropyl); 21.5 (OCH₃); 12.5 (CH₃, C-5 CH₃). 1D Diff. nOe shows 1.6% nOe enhancement for H6-H5′ and no other nOes expected between other endocyclic-sugar protons and H6 as found for the β-anomer (see below). (3a): ¹H-NMR (CDCl₃): 9.2 (s, 1H), NH; 7.71 (d, J=8.2 Hz, 2H), 4-toluoyl; 7.5 (s, 1H), H-6; 7.18 (d, J=7.9Hz, 2H),4-toluoyl; 5.44 (d, J_{3′,4′}=6.2Hz, 1H), H-3′; 4.87 (d, 1H) H-4′; 4.85-4.82 (m, 1H), H-5′; 4.65 (dd, J_{gem}=12.6 Hz, J_{5′,6′}=2.4Hz, 1H), H-6′; 4.3-4.2 (m, J_{5′,6″}=3.7 Hz, 2H), H-6″& H-1′; 3.8 (dd, J_{1″,1′-OH=}6.4Hz, J_{gem}=12.4 Hz, 1H), H-1″; 3.27 (t, 1H), 1′-OH, 2.4 (s, 3H), CH₃, 4-toluoyl; 1.6 (s, 1H), CH₃ (thymine); 1.56, 1.4 (s, 2×3H), CH₃, isopropyl. ¹³C-NMR (CDCl₃): 165.6 (C═O, 4-toluoyl); 164.3 (C-4); 150.1 (C-2); 144.6 (4-toluoyl); 137.3 (C-6); 129.2, 128.9, 125.9 (4-toluoyl); 113.4 (C-5); 108.6 (C—Me₂), 101.2 (C-2′); 86.1 (C-3′); 83.4 (C-5′); 81.7 (C-4′); 64.2 (C-6′); 63.7 (C-1′); 25.6, 24.1 (CH₃, isopropyl); 21.4 (CH₃, 4-toluoyl); 11.9 (CH₃, thymine). 1D Diff. nOe shows 0.21% nOe enhancement for H6-H6′, 0.08% nOe for H6-H3′ and 0.4% nOe for H6-H4′ which are consistent for a β-anomer.

1-(1′-O-Methanesufonyl-3′,4′-O-isopropyliene-6′-O-[4-toluoyl]-β-D-psicofuranosyl) thymine (5a).

Compound 3a (1.6 g, 3.5 mmol) was coevaporated with pyridine 3 times and dissolved in 25 ml of the same solvent. The mixture was cooled in an ice bath and methanesulfonyl chloride (0.75 ml, 9.7 mmol) was added dropwise to the mixture, continued the stirring for 15 min at the same temperature. The reaction was kept in at 4° C. for 12 h, then poured into cold saturated sodium bicarbonate solution and extracted with DCM. The organic phase was washed with brine, dried over MgSO₄, filtered, evaporated and coevaporated with toluene giving compound 5a (1.89 g, 3.6 mmol, 98%). R_{f}: 0.7 (System B). ¹H-NMR (CDCl₃): 7.75 (d, J=8.3 Hz, 1H), 4-toluoyl; 7.38 (d, J=1.3 Hz, 1H), H-6; 7.22 (d, J=8.4 Hz, 1H); 4-toluoyl; 5.39 (d, J_{3′,4′}=6 Hz, 1H), H-3′; 4.96 (d, J_{gem}=11.4 Hz, 1H), H-1′a; 4.94-4.88 (m, 2H), H4′ & H-5′; 4.7 (dd, J_{gem}=12.6 Hz, J_{5′,6′}=2.5 Hz, 1); H-6′; 4.39 (d, 1H), H-1″; 4.3 (dd, _{5′, 6″}=3.4 Hz, 1H), H-6″; 2.98 (s, 3H), CH₃; OMs; 2.4 (s, 3H), CH₃, 4-toluoyl; 1.7, 1.66 (s, 2×3H), CH₃, isopropyl. ¹³C-NMR (CDCl₃): 165.7 (C═O, 4-toluoyl); 162.9 (C-4); 150.2 (C-2); 145.1 (4-toluoyl); 135.5 (C-6); 129.1, 128.7, 125.6, (4-toluoyl); 114.2 (C-5); 110.1 (C—Me₂); 98.3 (C-2′); 87.1 (C-3′); 84.2 (C-5′); 81.7 (C-4′); 69.9 C-1′); 64.1 (C-6′); 37.4 (CH₃, 4-toluoyl); 25.8, 24.3 (CH₃, isopropyl); 21.3 (CH₃, mesyl); 12.3 (CH₃, thymine)

1-(1′-O-Methanesufonyl-6′-O-[4-toluoyl]-β-D-psicofuranosyl)thymine (5a).

Compound 4a (1.9 g, 3.5 mmol) was stirred with 10.5 ml of 90% CF₃COOH in water for 20 min at r.t. The reaction mixture was evaporated and coevaporated with pyridine. The residue on chromatography furnished 5a (1.58 g, 3.3 mmol, 92.5%). R_{f}: 0.3 (System B). ¹H-NMR (CDCl₃+CD₃OD): 7.75 (d, J=8.3 Hz, 1H), 4-toluoyl; 7.52 (d, J=1.24 Hz, 1H), H-6; 7.2 (d, J=8.4 Hz, 1H), 4-toluoyl; 4.81 (d, J_{gem}=11.6 Hz, 1H), H-1′; 4.76 (d, J_{3′,4′}=5.3 Hz, 1H), H-3′; 4.75 (dd, J_{gem}=12.6 Hz, J_{5′,6′}=3.5 Hz, 1H), H-6′; 4.62 (dt, 1H), H-5′; 4.58 (d, 1H); H-1′, 4.41 (dd, J_{4′,5′}=3 Hz, 1H), H-4′; 4.33(dd, 1H), H-6″; 2.98 (s, 3H), CH₃, OMs; 2.4 (s, 3H), CH₃, 4-toluoyl; 1.73 (s, 3H), CH₃, (thymine). ¹³C-NMR (CDCl₃+CD₃OD): 165.9 (C═O, 4-toluoyl), 163.8 (C-4), 151.7 (C-2); 144.9 (4-toluoyl); 136.3(C-6); 129.2, 129, 126.1 (4-toluoyl); 110.4 (C-5); 97 (C-2′); 83.9 (C-5′); 79.8 (C-3′); 72.2 (C-4′); 69.3 (C-1′); 63 (C-6′), 37.5 (CH₃, 4-toluoyl); 21.3 (CH₃, mesyl); 11.9 (CH₃, thymine)

1-(1′,3′-O-Anhydro-6′-O-[4-toluoyl]-β-D-psicofuranosyl)thymine (6a).

To a stirred solution of 80% NaH (171 mg, 5.7 mmol) in 15 ml of DMF in an ice bath, solution of compound 5a (1.3 g, 2.6 mmol) in 15 ml of DMF was added dropwise. The reaction mixture was stirred at the same temperature for 9 h, quenched with 10% acetic acid solution in water and evaporated. The residue was coevaporated with xylene and on chromatography yielded 6a (602 mg, 1.5 mmol, 60%). R_{f}: 0.5 (System C). ¹H-NMR (CDCl₃): 7.93 (d, J=8.1 Hz, 2H) 4-toluoyl; 7.25 (d, J=7.9 Hz, 2H) 4-toluoyl; 6.81 (s, 1H) H-6; 5.47 (d, J_{3′,4′}=3.9 Hz, 1H) H-3′; 5.15 (d, J_{gem}=7.9 Hz, 1H) H-1′; 4.79-4.72 (m, J_{gem}=12.3 Hz, J_{6′,5′}=2.55 Hz, 2H) H-1′ & H-6′; 4.55-4.42 (m, J_{6″,5′}=2.9 Hz, J_{4′,5′}=8 Hz, 3H), H-4′, H-5′, H-6″; 2.4 (s, 3H), CH₃, 4-toluoyl, 1.8 (s, 3H) CH₃, thymine. ¹³C-NMR (CDCl₃): 166.6 (C═O, 4-toluoyl), 164.3 (C-4); 149.2 (C-2); 143.8 (4-toluoyl); 135.1 (C-6); 129.5, 128.8, 126.5 (4-toluoyl); 111.6 (C-5); 90.9 (C-2′); 87.3 (C-3′); 80.9 (C-5′); 78.1 (C-1′); 70.3 (C-4′); 63 (C-6′); 21.2 (CH₃, 4-toluoyl); 11.8 (CH₃, thymine)

1-(1′,3′-O-Anhydro-β-D-psicofuranosyl)thymine (7a).

Compound 6a (570 mg, 1.5 mmol) was dissolved in methanolic ammonia (50 ml) and stirred at room temperature for 2 days. The solvent was evaporated and the residue on chromatography afforded 7a (378 mg, 1.4 mmol, 96%) R_{f}: 0.3 (System D) ¹H-NMR (CD₃OD, 600 MHz): 7.38 (d, J=1.25 Hz, 1H), H-6; 5.58 (d, J_{3′,4′}=3.8 Hz, 1H), H-3′; 5.33 (d, J_{gem}=8.1 Hz, 1H), H-1′; 4.9 (d, 1H), H-1″; 4.46-4.41(m, J_{4′,5′}=8.4 Hz, J_{5′,6′}=2.2 Hz, J_{5′,6″}=5.24 Hz, 2H), H-4′ & H-5′; 4.11 (dd, J_{gem}=12.4 Hz, 1H), H-6′; 3.9 (dd, 1H), H-6″; 2.1 (s, 1H), CH₃, (thymine). ¹³C-NMR (CD₃OD): 166.8 (C-4); 151.7 (C-2); 138.4 (C-6); 112.7 (C-5); 93.2 (C-2′), 89.3 (C-3′); 85.3 (C-5′); 79.9 (C-1′); 71.9 (C-4′); 62.7 (C-6′); 12.1 (CH₃, thymine).

1-(1′,3′-Anhydro-6′-O-dimethoxytrityl-β-D-psicofuranosyl)thymine (8).

To a solution of 7a (353 mg, 1.3 mmol) in anhydrous pyridine (6 ml) was added 4,4′-dimethoxytrityl chloride (510 mg, 1.15 mmol), and the mixture was stirred at r.t overnight. Saturated NaHCO₃ solution was added and extracted with dichloromethane. The organic phase was washed with brine, dried over MgSO₄, filterd and evaporated. The residue on column chromatography afforded 8 (647 mg, 1.13 mmol, 87%). R_{f}: 0.5 (System B). ¹H-NMR(CDCl3): 7.4-7.1 (m, 12H), arom (DMTr)& H-6; 6.85-6.82 (m, 4H), arom (DMTr); 5.4 (d, J_{3′,4′}=4.1 Hz, 1H), H-3′; 5.13 (d, J_{gem}=7.9 Hz, 1H), H-1′; 4.76 (d, 1H), H-1″; 4.35 (dd, J_{4′,5′}=8.3 Hz, 1H), H-4′; 4.28-4.21(m, J_{5′,6′}=2.5 Hz, J_{5′,6″}=4.7 Hz, 1H), H-5′; 3.98 (dd, J_{gem}=12.4 Hz, 1H), H-6′; 3.81 (dd, 1H), H-6″; 3.8 (s, 6H), OCH₃, DMTr; 1.92 (s, 3H), CH₃, thymine. ¹³-NMR (CDCl₃): 164.23, 158.1 (C-4); 149.5; 144.5 (C-2); 135.9, 135.3, 129.8, 128.9, 127.9, 127.5, 126.4, 112.8, (DMTr); 111.6 (C-5); 90.9 (C-2′); 87.6 (C-3′); 83.6 (C-5′); 78.2 (C-1′); 69.7 (C-4′); 60.8 (C-6′); 54.9 (DMTr); 11.9 (CH₃, thymine).

1-(1′,3′-Anhydro-6′-O-dimethoxytrityl-β-D-psicofuranosyl)thymine-4′-O-(2-cyanoethyl)-(N,N-diisopropyl)phosphoramidite (9a).

To a stirred solution of 8 (529 mg, 0.9 mmol) in 5 ml THF, 0.8 ml of N,N-diisopropyl ethyl amine was added under nitrogen atmosphere and stirred at r.t for 10 min. To this solution 2-cyanoethyl-N,N-diisopropyl phosphoramidochloride (0.4 ml, 1.8 mmol) was added and continued the stirring for 2 h. The reaction was quenched with methanol (3 ml) and the mixture was dissolved in DCM, washed with saturated NaHCO₃ solution and brine. The organic layer was dried over MgSO₄, filterd and evaporated. The residue on chromatography (30-40% EtOAc, cyclohexane+2% Et₃N) furnished 9a (632 mg, 0.81 mmol, 90%) R_{f}: 0.5 (system B) The compound was dissolved in DCM 3 ml) and precipitated from hexane at −40° C. ³¹P-NMR (CDCl₃):150.55; 150.46.

Synthesis, Deprotection and Purification of Oligonucleotides.

All oligonucleotides were synthesizesd on 1 μmol scale with 8-channel Applied Biosystems 392 DNA/RNA synthesizer. Synthesis and deprotection of AONs as well as RNA target were performed as previously described.¹⁸ For modified AONs fast depropecting amidites were used and they were deprotected by room temperature treatment of NH₄OH for 16 h. All AONs were purified by reversed-phase HPLC eluting with the following systems: A (0.1 M triethylammonium acetate, 5% MeCN, pH 7) and B (0.1 M triethylammonium acetate, 50% MeCN, pH 7). The RNA target was purified by 20% 7 M urea polyacrylamide gel electrophoresis and its purity and of all AONs (greater than 95%) was confirmed by PAGE. Representive data from MALDI-MS analysis: AON (4) [M−H]⁻ 4478.7; calcd 4478; RNA target (7) [M−H]⁻ 4918.1; calcd 4917.1.

1-(1′,3′-O-Anhydro-β-D-psicofuranosyl)uracil (7b)

¹H-NMR(CD₃OD): 7.48 (d, J_{5,6}=8 Hz, 1H, H-6), 5.81(d, 1H, H-5), 5.49 (d, J_{3′,4′}=3.1 Hz, 1H, H-3′),5.24 (d, J_{gem}=8 Hz, 1H, H-1′), 4.8 (d, 1-H, H-1″), 4.38-4.3 (m, J_{4′,5′}=8.1 Hz, J_{5′,6′}=1.6 Hz, J_{5,6″}=6 Hz, 2H, H-4′ and H-5′), 4.04 (dd, J_{gem}=13 Hz, 1H, H-6′), 3.83 (dd, 1H, H-6″). ¹³C-NMR (CD₃OD): 166.4 (C-4), 151.4 (C-2), 143 (C-6), 103.6 (C-5), 93 (C-2′), 89.3 (C-3′), 85.4 (C-5′), 79.9 (C-1′), 71.8 (C-4′), 62.6 (C-6′).

1-(1′,3′-O-Anhydro-β-D-psicofuranosyl)cytosine (7c).

¹H-NMR(D₂O): 7.28 (d, J_{5,6}=7.3 Hz, 1H, H-6), 5.94 (d, 1H, H-5), 5.44 (d, J_{3′,4′}=3.1 Hz, 1H, H-3′), 5.14 (d, J_{gem}=8.3 Hz, 1H, H-1′), 4.76 (d, 1-H, H-1″), 4.29-4.23 (m, J_{5′,6″}=4.9 Hz, 2H, H-4′ and H-5′), 3.9 (d, J_{gem}=12.3 Hz, 1H, H-6′), 3.74 (dd, 1H, H-6″). ¹³C-NMR (D₂O): 166.5 (C-4), 156.1 (C-2), 141.9 (C-6), 96.4 (C-5), 91.8 (C-2′), 87.5 (C-3′), 82.6 (C-5′), 78.7 (C-1′), 69.6 (C-4′), 60.5 (C-6′).

RNase H Digestion Assays

DNA/RNA hybrids (0.8 μM) consisting of 1:1 mixture of antisense oligonucleotide and target RNA (specific activity 50000 cpm) were digested with 0.3 U of RNase H in 57 mM Tris-HCl; (pH 7.5), 57 mM KCl, 1 mM MgCl₂ and 2 mM DTT at 21-37° C. Prior to the addition of the enzyme reaction components were preannealed in the reaction buffer by heating at 80° C. for 4 min followed by 1.5 h. equilibration at 21-37° C. Total reaction volume was 26 μl. Aliquots (7 μl) were taken after 5, 15, 30, 60 and 120 min and reaction was stopped by addition of the equal volume of 20 mM EDTA in 95% formamide. RNA cleavage products were resolved by 20% polyacrylamide denaturing gel electrophoresis and visualized by autoradiography. Quantitation of cleavage products was performed using a Molecular Dynamics PhosphorImager. The experiment is repeated at least 4 times and average values of the % of cleavage are reported here.

Exonuclease Degradation Studies

Stability of the AONs towards 3′-exonucleases was tested using snake venom phosphodiesterase from Crotalus adamanteus. All reactions were performed at 3 μM DNA concentration (5′-end ³²P labeled with specific activity 50000 cpm) in 56 mM Tris-HCl (pH 7.9) and 4.4 mM MgCl₂ at 22° C. Exonuclease concentration of 70 ng/μl was used for digestion of oligonucleotides (total reaction volume was 16 μl). Aliquots were quenched by addition of the same volume of 20 mM EDTA in 95% formamide. Reaction progress was monitored by 20% 7 M urea PAGE and autoradiography.

Endonuclease Degradation Studies

Stability of AONs towards endonuclease was tested using DNase 1 from Bovine pancreas. Reactions were carried out at 0.9 μM DNA concentration (5′-end ³²P labeled with specific activity 50 000 cpm) in 100 mM Tris-HCl (pH 7.5) and 10 mM MgCl₂ at 37° C. using 30 unit of DNase 1 (total reaction volume was 22 μl). Aliquots were taken at 60, 120, 180 and 240 min and quenched with the same volume of 20 mM EDTA in 95% formamide. They were resolved in 20% polyacrylamide denaturing gel electrophoresis and visualized by autoradiography.

1. Modified nucleosides and nucleotides, represented by the following formula: 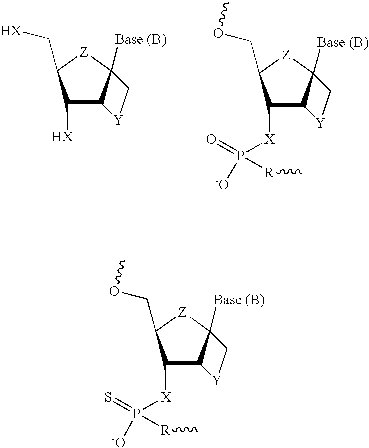
wherein combinations of modifications with X, Y, Z, R or B are claimed:
X=O or S, or NH or NCH₃, CH₂ or CH(CH₃),
Y=O, S, or NH or NCH₃, CH₂ or CH(CH₃);
Z=O, S, or NH or NCH₃, CH₂ or CH(CH₃)
R=O or S, or NH or NCH₃, CH₂ or CH(CH₃)
B=A, C, G, T, U, 5-F/Cl/BrU or —C, 6-thioguanine, 7-deazaguanine;
α- or β-D- (or L) ribo, xylo, arabino or lyxo configuration
and oligonucleotides and oligonucleosides comprising these.

2. Reagents for the preparation of modified nucleoside-nucleotide analogs, oligonucleotides or oligonucleosides by solid or solution phase synthesis: 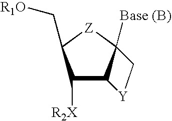
wherein combinations of modifications with Y, Z, R or B are claimed:
X=O or S, or NH or NCH₃, CH₂ or CH(CH₃),
Y=O, S, or NH or NCH₃, CH₂ or CH(CH₃);
Z=O, S, or NH or NCH₃, CH₂ or CH(CH₃)
R=O or S, or NH or NCH₃, CH₂ or CH(CH₃)
B=A, C, G, T, U, 5-F/Cl/BrU or —C, 6-thioguanine, 7-deazaguanine;
α- or β-D- (or L) ribo, xylo , arabino or lyxo configuration
comprising the following possible variations of the building blocks for synthesis of compounds:
for Uracil:
N3-Benzoyl, N3-(4-toluoyl), N3-(2-toluoyl), N3-(4-anisoyl); N3-(4-chlorobenzoyl), N3-(2,2,2-tichloro-t-butyloxycarbonyl), N3-(triphenylmethanesulfenyl), N3-(butylthio-carbonyl), N3-(methoxyethoxymethyl), O4-(2-Nitrophenyl), O4-(2-(4-cyanophenyl)-ethyl), O4-(2-(4-nitrophenyl)-ethyl), O4-phenyl, O4-2-methylphenyl, O4-(4-methylphenyl), O4-(2,4-di-methylphenyl), O4-(3-chlorophenyl), O4-(2-(4-nitrophenylsulfonyl)-ethyl), O4-(6-methyl-3-pyridyl), O4-(4-nitrophenylethoxycarbonyl), O4-(4-methyl-3-pyridyl), O4-2,4,6-trimethylphenyl,
for Cytosine:
N4-Anisoyl, N4-benzoyl, N4-(3,4-dimethylbenzoyl), N4-acetyl, N4-phenoxyacetyl, N4-dimethylaminomethylene, N4-benzyloxycarbonyl, N4-levulinoyl, N4-isobutyryl, N4-(2-nitrophenylsulfenyl), N4-isobutoxycarbonyl, N4-(2,2,2-trichloro-t-butyloxycarbonyl), N4-(9-fluorenylmethoxycarbonyl), N4-(N-methyl-2-pyrrolidine amidine), N4-(N,N-di-n-butylformamidine), N4-(3-methoxy-4-phenoxybenzoyl), N4-(isopropoxyacetyl), N4-(2-(tertbutyldiphenylsilyloxymethyl)-benzoyl), N4-(phenylsulfonylethoxycarbonyl), N4-(4-chlorophenylsulfonylethoxycarbonyl), N4-(2-chlorophenylsulfonylethoxycarbonyl), N4-(4-nitrophenylethoxycarbonyl), N4-2-(acetoxymethyl)benzoyl,
for Adenine:
N6-Di-n-butylformamidine, N6-benzoyl, N6-succinyl, N6, N6-phthaloyl, N6-(4,5-dichlorophthaloyl), N6-tetrachlorophthaloyl, N6-(2-(4-nitrophenyl)-ethoxycarbonyl), N6-phenoxyacetyl, N6-(9-fluorenylmethoxycarbonyl), N6-(3-chlorobenzoyl), N6-anisoyl), N6-(4-tertbutylbenzoyl), N6-phenoxycarbonyl, N6-benzyloxycarbonyl, N6-isobutoxycarbonyl, N6-(2,2,2-trichloro-t-butyloxycarbonyl), N6-dimethylacetamidine, N6-(2-nitrophenylsulfenyl), N6-dimethylaminomethylene, N6-di-n-butylaminomethylene, N6-(N-methyl-2-pyrrolidine amidine), N6-(N,N-di-n-butylformamidine), N6-(3-methoxy-4-phenoxybenzoyl), N6-isopropoxyacetyl, N6-(2-(tertbutyldiphenylsilyloxymethyl)-benzoyl), N6-phenylsulfonylethoxycarbonyl, N6-(4-chlorophenylsulfonylethoxycarbonyl), N6-(2-chlorophenylsulfonylethoxycarbonyl), N6-(4-nitrophenylethoxycarbonyl), N6-2-(acetoxymethyl)benzoyl, N6-(m-chlorobenzoyl).
for Guanine and 7-deazaguanine (hypoxanthine has the same O6 protection as guanine or 7-deazaguanine):
N2-Isobutyryl, acetyl, N2-(4-tertbutylbenzoyl), N2-benzyloxycarbonyl, N2-phenoxyacetyl, N2-benzoyl, N2levulinoyl, N2-(2-nitrophenylsulfenyl), N2-(9-fluorenylmethoxycarbonyl), N2-(2,2,2-trichloro-t-butyloxycarbonyl), N2-propionyl, N2-dimethylaminomethylene, N2-dimethylacetamidine, N2-(N-methyl-2-pyrrolidineamidine), N2-(N,N-di-n-butyl-formamidine), N2-phenylacetyl, N2-(1,2-diisobutyryloxyethylene), N2-(3-methoxy-4-phenoxybenzoyl), N2-methoxyacetyl, chlorophenoxyacetyl, N2-isopropoxy-acetyl, N2-(2-(tertbutyldiphenylsilyloxymethyl)-benzoyl), N2-phenylsulfonylethoxycarbonyl, N2-(4-chlorophenylsulfonylethoxycarbonyl), N2-2-(acetoxymethyl) benzoyl, N.sup.2-(3,4-dichlorobenzoyl), O6-Benzyl, O6-(2-(4-nitrophenyl)-ethyl), O6-(2-nitrophenyl), O6-(4-nitrophenyl), O6-diphenylcarbamoyl, O6-(3,4-dimethoxybenzyl), O6-(3,5-dichlorophenyl), O6-(2-cyanoethyl), O6-(2-trimethylsilylethyl), O6-phenylthioethyl, O6-(4-nitrophenylthioethyl), O6-butylthiocarbonyl, O6-(6-methyl-3-pyridyl), O6-(2-(4-nitrophenylsulfonyl)-ethyl), O6-(4-methyl-3-pyridyl), N2-(4-nitrophenylethoxycarbonyl), O6-allyl or any combination of these protecting groups for O6, N2-bis protection,
for Thymine:
O4-phenyl, O4-(2-(4-nitrophenyl)-ethyl), O4-(2-(4-nitro phenylsulfonyl)-ethyl), O4-(2-methylphenyl), O4-(4-methyl phenyl), O4-(2,4-dimethylphenyl), N3-benzoyl, N3-(4-anisoyl), N3-(4-toluoyl), N3-(2-toluoyl).
R₁=5′-protecting group such as:
9-Fluorenylmethoxycarbonyl, 4-chlorophenylsulfonylethoxy carbonyl, 4-nitrophenylsulfonyl-ethoxycarbonyl, phenyl sulfonylethoxycarbonyl, 2,2,2-tribromoethoxycarbonyl, levulinyl, 4,4′,4″-tris(4,5-dichlorophtalimide)trityl, 4,4′,4″-tris(benzoyloxy)trityl, 4,4′,4″-tris(levulinyl oxy)trityl, p-anisyl-1-naphtylphenylmethyl, di-p-anisyl-1-naphtylmethyl, p-tolyldiphenylmethyl, 3-(imidazolylmethyl)-4,4′-dimethoxytrityl, methoxyacetyl, chloroacetyl, phenoxyacetyl, 4-chlorophenoxyacetyl, trityloxyacetyl, .beta.-benzoylpropionyl, isobutyloxycarbonyl, 4-nitrobenzyloxy carbonyl, 2-(methylthiomethoxymethyl)-benzoyl, 2-(iso propylthiomethoxymethyl) benzoyl, 4-(methylthiomethoxy butyryl, p-phenylazophenyloxycarbonyl, 2,4-dinitrophenyl ethoxycarbonyl, pivaloyl, 2-dibromomethylbenzoyl, tert-butyldimethylsilyl, 4,4′-dimethoxytrityl, 4′-monomethoxy trityl, 4-decyloxytrityl, 4-hexadecyloxytrityl, trityl, 1,1-bis-(4-methoxyphenyl)-1′-pyrenyl, 9-phenylxanthen-9-yl, 9-phenylthioxanthen-9-yl, 7-chloro-9-phenylthioxan then-9-yl, 9-(4-methoxyphenyl)-xanthen-9-yl, 9-(4-octadecyloxyphenyl)-xanthen-9-yl
R₂=3′-phosphate, 3′-(H-phosphonate), 3′-phosphoramidate, 3′-phosphoramidite, 3′-(alkanephosphonate) such as:
(a) 3′phosphate: 2,2,2-Trichloroethyl, 2,2,2-tribromoethyl, 2-cyanoethyl, benzyl, 4-chlorophenyl, 4-nitrophenylethyl, 2-chlorophenyl, 2-diphenylmethylsilyl ethyl, phenylthio;
(b) 3′-phosphate esters: 2-Cyanoethyl-4-chlorophenyl, 2-cyanoethyl-2-cyanoethyl, 2-cyanoethyl-2-chlorophenyl, phenylsulfonylethyl-2-chlorophenyl, 9-fluorenylmethyl-2-chlorophenyl, 9-fluorenylmethyl-4-chlorophenyl, phenylsulfonylethyl-4-chlorophenyl, phenylsulfonylethyl-2-chloro phenyl, 2,2,2-tribromoethyl-4-chlorophenyl, 2,2,2-tribromo ethyl-2-chlorophenyl, 2,2,2-trichloroethyl-4-chlorophenyl, 2,2,2-trichloroethyl-2-chlorophenyl, 2-cyanoethyl-2-chloro-4-tritylphenyl, 2,2,2-tribromoethyl-2-chloro-4-tertbutyl phenyl, 4-nitrophenyl-phenyl, 2,4-dinitrobenzyl-2-chloro phenyl, 2,4-dinitrobenzyl-4-chlorophenyl; S,S-diphenyl phosphorodithioate; 2-chlorophenyl-phosphoranilidate, phenyl-phosphoranilidate,
(c) 3′-halophosphites (chloro or bromo): phenylsulfonylethoxy, methylsulfonylethoxy, 2-(isopropyl sulfonyl)-ethoxy, 2-(tertbutylsulfonyl)-ethoxy, benzyl sulfonylethoxy, 4-nitrobenzylsulfonylethoxy, 9-fluorenyl methoxy, 2-(4-nitrophenyl)-ethoxy, methoxy, 2-cyano-1,1-dimethylethoxy, 2,2,2-trichloro-1,1-dimethylethoxy, 2,2,2-trichloroethoxy, 2-cyanoethoxy, 2-cyano-1-methylethoxy, 2-cyano-1,1-dimethylethoxy-, 2-(4-nitrophenyl)-ethoxy, 2(2-pyridyl)-ethoxy, 2-methylbenzyloxy, 4-chlorobenzyloxy, 2-chlorobenzyloxy, 2,4dichlorobenzyloxy, 4-nitrobenzyloxy, allyloxy, phenoxy, 4-nitrophenoxy, pentafluorophenyoxy, pentachlorophenoxy, 2,4,5-trichlorophenoxy, 2-bromophenoxy, 4-bromophenoxy, 2-methylphenoxy, 2,6-dimethylphenoxy, 2,4-nitrophenoxy, 1,1,1,3,3,3-hexafluoro-2-propyloxy, 2-chlorophenoxy.
(d) 3′-phosphoramidites: phenylsulfonylethoxydimethylamino, methylsulfonylethoxy-morpholino, 2-(isopropylsulfonyl)-ethoxy-morpholino, 2-(tertbutylsulfonyl)-ethoxy-morpholino, benzylsulfonylethoxy-morpholino, 4-nitrobenzylsulfonylethoxy-morpholino, 9-fluorenylmethoxymorpholino, 2-(4-nitrophenyl)-ethoxy-morpholino, 2-(4-nitrophenyl)-ethoxy-hexahydroazepine, 2-(4-nitrophenyl)ethoxy-octahydrazonine, 2-(4-nitrophenyl)-ethoxyazacyclo tridecane, methoxy-pirrolidino, methoxy-piperidino, methoxy-diethylamino, methoxy-diisopropilamino, methoxy-2,2,6,6-tetramethyl-N-piperidino, methoxy-morpholino, 2-cyano-1,1-dimethylethoxy-morpholino, 2,2,2-trichloro-1,1-dimethylethoxydimethylamino, 2,2,2-trichloro-1,1-dimethyl ethoxydiethylamino, 2,2,2-trichloro-1,1-dimethylethoxy-diisopropylamino, 2,2,2-trichloro-1,1-dimethylethoxymorpholino, 2,2,2-trichloroethoxydimethylamino, 2-cyanoethoxy diethylamino, 2-cyanoethoxydiisopropylamino, 2-cyanoethoxy morpholino, 2-cyano-1-methylethoxy-diethylamino, 2-cyano-1,1-dimethylethoxydiethylamino, 2-cyano- 1,1-dimethylethoxy -diisopropylamino, methylsulfonylethoxydiethylamino, methylsulfonylethoxydiisopropylamino, 2,2,2-trichloroethoxydiisopropylamino, 2,2,2-trichloro-1,1-dimethylethoxydiisopropylamino, 2-(4-nitrophenyl)-ethoxy-diisopropyl amino, 2(2-pyridyl)-ethoxy-diisopropylamino, 2(4-pyridyl)ethoxydiisopropylamino, 2-methylbenzyloxy-diisopropyl amino, 4-chlorobenzyloxy-diisopropylamino, 2-chlorobenzyl oxydiisopropylamino, 2,4-dichlorobenzyloxy-diisopropyl amino, 4-nitrobenzyloxydiisopropylamino, allyloxydiiso propylamino, allyloxydimethylamino, phenoxydiethylamino, 4-nitrophenoxydiethylamino, pentafluorophenoxydiethyl amino, pentachlorophenoxy-diethylamino, 2,4,5-trichloro phenoxydiethylamino, 2-bromophenoxydiethylamino, 4-bromophenoxydiethylamino, 2-methylphenoxydiethylamino, 2,6-dimethylphenoxydiethylamino, 2,4-nitrophenoxy diethylamino, 1,1,1,3,3,3-hexafluoro-2-propyloxydiiso propylamino, 2-chlorophenoxy-morpholino, bis(diisopropyl amino), bis(diethylamino), bis(morpholino).
(e) phosphonate: methyl, ethyl, trifluoromethyl, cyanoethyl, trichloroethyl, tribromoethyl, trifluorethyl.

3. Therapeutic composition comprising the modified oligonucleotides and oligonucleosides according to claim 1 together with physiologically acceptable carriers.

4. A method for antisense therapy, comprising administration of the therapeutic composition according to claim 4 to a patient in need thereof.

5. A method according to claim 5, wherein the antisense therapy is against oncogenic sequences.

6. A method according to claim 5, wherein the antisense therapy is against pathogenic sequences.

7. A method according to claim 5, wherein the antisense therapy is for treatment of genetic disorders.

8. A diagnostic kit comprising the modified oligonucleotides and oligonucleosides according to claim 1.

9. A method of diagnosing nucleotide polymorphism(s) in an individual, comprising use of the diagnostic kit according to claim 8.

10. A DNA sequencing kit comprising the modified nucleosides and nucleotides. 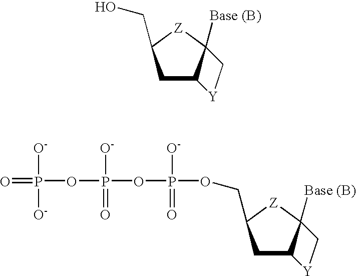
wherein combinations of modifications with Y, Z, or B are claimed:
Y=O, S, or NH or NCH₃, CH₂ or CH(CH₃);
Z=O, S. or NH or NCH₃, CH₂ or CH(CH₃)
B=A, C, G, T, U, 5-F/Cl/Br-U; 7-deaza-G or hypoxanthine
α- or ,β-D- (or L) ribo, xylo, arabino or lyxo configuration

11. Use of the nucleotides and nucleosides according to claim 1 for production of aptamers.

12. Use of the compounds according to claims 1, 2 and/or 10 for drug development.

13. Use of the compounds according to claims 1, 2 and/or 10 in any form of polymerase chain reaction (PCR).

14. Use of the compounds according to claims 1, 2 and/or 10 in any molecular biology kit for diagnosis, detection or as reagent.
